(19) United States
(12) Patent Application Publication
(10) Pub. No.: US 2008/0088629 A1
(43) Pub. Date: Apr. 17, 2008

(54) PREVENTIVE MODULE FOR AVOIDING DISEASES

(76) Inventors: Gunther Lorenz, Frankenthal (DE); Oliver Mast, Freinsheim (DE)

Correspondence Address: ROCHE DIAGNOSTICS OPERATIONS INC., 9115 Hague Road, Indianapolis, IN, 46250-0457, US

(21) Appl. No.: 11/937,236

(22) Filed: Nov. 8, 2007

Related U.S. Application Data
(63) Continuation of application No. PCT/EP2006/001347, filed on Feb. 15, 2006.

(30) Foreign Application Priority Data
May 11, 2005 (DE) 202005007461
May 11, 2005 (DE) 102005021779.6

(51) Int. Cl. G06T 11/20; A61B 5/00

(52) U.S. Cl. 345/440.2; 600/300; 345/440

(57) ABSTRACT

Embodiments of a system are described for visualizing the state of health of a patient, comprising an input unit for reading in patient data, an evaluation unit for evaluating the data and an output unit which outputs the results of the evaluation in a graphical form. The evaluation unit contains a program which is configured to read in a parameter value of the patient for at least one risk parameter, to calculate a current risk status, and to calculate a short-term target risk for a change of the parameter value.

The present application is a continuation of PCT Application No. PCT/EP2006/001347, filed Feb. 15, 2006, which is based on and claims priority to German Application Nos. DE 202005007461.6 and DE 102005021779.6, both filed on May 11, 2005 which are hereby incorporated by reference in their entireties.

### BACKGROUND

The invention concerns the visualization of patient-related data as a representation of his or her state of health.

There has been a continuous to and fro in the discussion about the importance of preventive medicine. Even if preventive medicine is not always accepted due to short-term considerations, the conclusion from long-term considerations is that prevention is a more cost-efficient solution. Moreover, an additional advantage of preventive medicine is that it results in an improvement in the quality of life of many patients. In the case of serious diseases preventive medicine is often the only possibility to heal such diseases. Thus for example many types of cancer (of the lung, ovaries or breast) and also stroke are diseases which, without preventive measures, can rapidly end in death. In contrast the life of many millions of people can be saved or substantially prolonged in the case of early recognition in combination with preventive measures.

The development of preventive medicine has produced results in many areas of research. There is a vast amount of publications, clinical studies and experiments which have led to the development of preventive measures for the above-mentioned diseases and also many other diseases. It is completely impossible for the patient to distinguish between reliable and unreliable studies among this large quantity of information and to use it to make an assessment about the use of preventive measures for his or her own health. For this reason it is advisable to give the doctor and patient tools which make calculations based on scientifically-founded studies which propose preventive measures that are individually tailored to the patient.

For example diabetes mellitus is a clinical picture the importance of which is underscored by the risk of secondary diseases. Diabetes mellitus is a complex pathological process. Diagnostic and therapeutic decisions should be evidence-based provided sufficient scientific findings are available. It is hardly possible to manage the flood of data generated by medical research in order to use it for treatment without a systematic decision support.

Prior art health monitoring programs are described in U.S. Patent Application No. 2004/0122715 and issued U.S. Pat. No. 6,584,445.

What is absent in prior art systems is a visualization of the prognosis for the patient if he or she were to change certain ways of life or if he or she would undergo certain treatments. Without these perspectives there is no incentive for the patient to change his or her way of life. This is an important component in the treatment of patients who have to reckon with secondary diseases as a result of a disease such as diabetes.

Furthermore, it is not apparent from prior art systems what effect a change in parameters that can be influenced by the patient would have.

In light of the shortcomings of the prior art, it is an object to provide a system which offers the patient additional information on his or her possible state of health when he or she changes parameter values of at least one risk parameter by changing, for example, his or her way of life.

Another object is that in subsequent examinations of the patient he or she can be informed about the direction in which his or her health has developed.

Another object is to provide a patient the opportunity of setting a new target value at any time for each individual risk parameter and then always be shown the previous results as a comparison in a visual form.

### SUMMARY OF EMBODIMENTS OF THE INVENTION

These objects and more are achieved according to the present invention disclosed herein by means of a system for visualizing the state of health of a patient, the system comprising an input unit for reading in patient data, an evaluation unit for evaluating the data and an output unit which outputs the results of the evaluation in a graphical form characterized in that the evaluation unit contains a program which reads in a parameter value of the patient for at least one risk parameter and calculates a current risk status and calculates a “target risk” (referred to in the following disclosure as short-term target risk) for a change of the parameter value.

In embodiments of the system described in the following disclosure, a current risk status is calculated on the basis of parameter values of the patient for various risk parameters. Afterwards a potential short-term target risk is calculated for the patient when individual parameter values are changed according to compliance with treatment or lifestyle changes. Hence in this case not only possible types of treatments and changes in life style are proposed but they are also directly related to the projected implications for the patient. Thus the patient is shown the direction in which he or she can develop when he or she changes certain parameter values. This is an additional incentive for the patient to change his or her lifestyle. The embodiments of the system according to the present invention further provide the patient a visual representation of the results using up to four different examination reports. Using such comparative graphics, the patient is able to recognize whether he or she can already discern an improvement in some risk parameters or whether he or she may have deteriorated. The comparison of the current state of risk with the short-term target risk also informs the patient about the magnitude of his or her calculated potential for further improvements.

Embodiments of the system further offer the possibility of carrying out risk assessments and displaying them graphically over the course of up to four different assessment times. In order to achieve this object, embodiments of a system are described which enable the state of health of a patient to be visualized consisting of an input unit to input patient data, an evaluation unit to evaluate the data and an output unit which outputs the results of the evaluation in a graphical form. The evaluation unit contains a program which inputs the profile of values for the risk parameters of the patient and calculates a current risk status from this profile and also calculates a short-term target risk under the assumption of a target profile determined by the treatment provider. As a guideline for the patient, an “ideal risk status” (in the following also referred to as long-term target risk) is added in a similar manner which shows the magnitude of the risk when the calculation is based on values that have been designated by the National Diabetes Care Guideline as threshold values to the low-risk range.

The profile of risk parameters typically comprises blood values such as long-term blood sugar or cholesterol as well as other patient-specific data such as blood pressure, smoking, weight, age and gender of the patient. In addition, patient related data available to the doctor or patient can be entered into the system. A patient-specific risk can then be calculated from these data for many different diseases. This is of particular interest for diabetic patients because their risk of contracting secondary diabetic diseases is very high. In this case it is possible for example to calculate the risks for cardiac infarction, stroke, kidney failure, loss of sight or foot amputation. The result of this calculation can be displayed graphically by the output unit according to a didactically prepared and scientifically evaluated concept.

The input unit in embodiments of the disclosed system can for example be a data carrier reading instrument, a scanner, a data interface, or any other known electronic input means. This allows all available electronic data and also data in a paper form to be read into the system. Of course data in a paper form can also be entered by the keyboard of an electronic system. The evaluation unit for processing the input data typically comprises a program which contains various forms of algorithms. The program evaluates the data that are present in an electronic form. Typically in the course of evaluation, the individual patient data are linked in the form determined by the algorithm with the medical findings present in the system which are derived from the relevant medical studies. The evaluated data are now passed electronically to an output unit. Typically, the output unit electronically generates a graphical report which can be printed out on a printer or can be sent as an electronic document. The output unit can also be another output unit known to a person skilled in the art. In this manner the results of the evaluation are visualized for the patient and for the doctor.

In one embodiment, in use, the program in the evaluation unit reads-in the current risk parameter profile of a patient in which each parameter has at least one parameter value and uses an algorithm to calculate the current risk status of the patient for this array of risk parameters. The value for the current patient risk (in percent) states how many persons from a group of 100 persons having the same medical profile as the patient concerned would statistically suffer from the respective secondary disease within the next 10 years. In order to calculate the short-term target risk of the patient, the patient or the doctor specifies the target array (which is usually agreed with the patient) with regard to the variable risk factors, and from this (or from the array specified by the National Diabetes Care Guideline for the relevant diabetes sequela) the system firstly calculates the absolute risk difference between the “current risk” and “short-term target risk”. Subsequently, the relative risk reduction (potential) is determined from this absolute risk difference based on the current risk.

The patient may or may not be able to influence the risk parameters that are entered into the system. For example, risk parameters such as smoking, blood pressure, total cholesterol value, HDL cholesterol value, long-term blood sugar and weight can be influenced by the patient. In contrast risk parameters such as age, gender, duration of the disease and anamnestic data cannot be influenced by the patient. Displaying the potential shows the patient his or her health prospects i.e. the proportion of the total risk which he or she himself can positively influence by changing his or her way of living and behavior pattern (lifestyle, therapy compliance, etc.). Thus, as a smoker he or she could give up smoking or as an overweight patient he or she could engage in more sport activities in order to influence the corresponding risk parameters and thus the risk for diabetes sequelae. If the patient is monitored over a longer time period, the patient can recognize at any time whether his or her current risk status has developed towards his or her short-term target risk or whether the current risk status has deteriorated. In this regard, the current risk status is calculated each time using the current values for the risk parameters.

For this the absolute probabilities of typical diabetic long-term sequelae occurring in the next 10 years according to model prognoses is calculated with reference to personal parameters and the current health status of the patient. This graphical information can for example be used in a doctor-patient discussion to illustrate to the patient the effects on health of an unhealthy life style and lack of cooperation in the therapy.

In certain embodiments, in order to demonstrate to the patient how he or she can influence the further course of the disease and thus the importance of active cooperation, health potentials are estimated using scenario calculations. The individual target values for the risk factors that have been agreed with the patient to calculate the short-term target risk or the threshold values recommended by the National Diabetes Care Guideline for calculating the long-term target risk are used as a standard for comparison. The estimation of these health potentials provides arguments for relating individual targets with the patient and their stepwise approximation to the guideline recommendations.

In order to use concrete therapeutic results to increase motivation, the development of the risk parameters and long-term secondary risks over time is illustrated by comparative graphics based on additional risk and potential reports (in the form of sequelae reports) that have been compiled over a sufficiently long period of treatment. This representation is configured to document if and how the health opportunities have been utilized. The system is further configured to calculate and visualize absolute and relative risk differences. The absolute risk difference is the calculated difference between the current risk of the patient and the reduced risk which he or she would have with improved risk parameter values. The relative risk difference (potential) relates this absolute risk difference to the current absolute risk. This, for example, might show that if the risk of a cardiac infarction would be 33 percent lower compared to the current risk when the target array of all risk factors that can be influenced is present, one out of three ensuing cardiac infarctions could be statistically avoided in this ideal array.

Hence the risk and potential report can help the patient or the doctor to positively influence the attitude of the patient towards his or her disease and his or her awareness for his or her own possibilities and chances.

In other embodiments, the risk and potential report gives the patient and/or doctor the opportunity to extend his or her experience with diabetes mellitus by the bundled empirical knowledge from more than 80 studies selected for their scientific quality, such as, e.g., UKPDS (UK Prospective Diabetes Study; Lancet 1998; 352 (9131): 837-853); DCCT (Diabetes control and complication trial; N. Engl. J. 1993, 329(14): 977-986) and to utilize the findings from these studies to support the therapy decision.

For example, in the case of diabetes, by linking the results of the studies with the master data of the patient and his or her individual diagnostic and anamnestic findings, a current individual risk profile for the five long-term sequelae of diabetes is estimated for the patient. Here, individual diagnostic and anamnestic findings may include (1) age, gender, duration of diabetes, smoking status (master data); (2) long-term blood sugar HbAlc, total cholesterol and HDL cholesterol; (3) systolic blood pressure; and (4) previous diseases. The five long-term sequelae of diabetes include cardiac infarction, stroke, foot amputation, kidney failure, and loss of sight.

For this purpose, disease courses are simulated using a diabetes model based on the patient-specific data. In addition health potentials are calculated with reference to the aspired goals with regard to metabolic adjustment, blood pressure and smoking status. These calculations take into account the individually agreed targets or the targets specified by the National Care Guideline as well as the current health situation as well as personal characteristics (master data) of the patient.

In this regard, embodiments of the system are configured to simulate the potential course of the disease for five typical long-term diabetic sequelae. In one embodiment, the complex overall structure of the model generated from such simulation is composed of model components for the individual long-term sequelae. In each model component a Markov state process with time-dependent and state-dependent transition probabilities depicts the progress of a secondary disease with its individual stages (health states). The model simulations are currently based on the results of about 80 published diabetes studies.

In order to continuously update the system of the present invention, the current literature in the fields of medicine, epidemiology and health economy should be regularly reviewed for new scientific findings. The publications gathered by a systematic literature search may be subjected to a multistep selection process. Firstly they may be qualitatively checked for relevance. If a study is regarded to be relevant, it may then be analysed on the basis of defined quality criteria (such as with regard to number of cases, study design etc.) and possibly also for systematic errors which could distort the study results and thus the conclusion (“bias”: is a systematic error which distorts study results), evaluated and classified into an evidence class (according to the MERGE-classification from: Methods for Evaluating Research Guideline Evidence in Harbour R, Miller J: A new system for grading recommendations in evidence based guidelines, BMJ 2001; 323: 334-336). The results of studies which in each case had the currently best MERGE classification (and consequently a low bias) are then incorporated into the disease model. MERGE stands for “Methods for Evaluating Research Guideline Evidence” and generates quality check lists which should essentially check the extent to which study results are influenced by external factors (“bias”). The study is allocated into an evidence class according to the degree of bias.

This selection and evaluation process is typically documented according to the requirement for building a suitable model. In the case of conflicting evidence, experts may be integrated into the process for deciding which studies should ultimately be used in the model.

The validity of model calculations can be ensured by validating the disease model as well as by other quality assurance measures such as determining defined patient inclusion and exclusion criteria and evidence-ensured ranges for the values of the risk parameters (e.g. for the age of the patient). Parameter values near to the evidence-based value range are replaced by the minimum or maximum values of the evidence range in order to allow an approximation calculation; values which deviate more strongly are excluded.

In embodiments of the system of the present invention, the value ranges of the National Health Care Guideline for Diabetes mellitus Type II (May 2002) are included, which is chaired by the Medical Centre for Quality Assurance on behalf of the German. Medical Association with the assistance of the Drug Commission of the German Medical Association (AkdÄ), the German Diabetes Society (DDG), the Specialists Commission for Diabetes in Saxony as well as the German Society for Internal Medicine (DGIM and the working group of scientific medical specialists societies (AWMF). This guideline has found abroad consensus in Germany. The system also offers the possibility of defining individual target value parameters (e.g. in the sense of intermediate targets) which deviate from the guideline which it contains and allows a representation of the optimization potential of the patient in relation thereto.

Certain embodiments of the system typically comprise three subcomponents:
a) server with model core (diabetes disease model)
b) modular client (data import, data exchange with the server, report generator)
c) risk and potential reports.

The server subcomponent typically comprises the disease model e.g. diabetes, a control logic and a data base. The diabetes disease model represents the core of the system and comprises an algorithm which represents a model of the structure of the disease Diabetes mellitus (differentiated into type 1 and type 2) based on important medical disease parameters. A distinction can be made between five submodels (myocardial infarction, stroke, kidney failure, loss of sight and amputation) corresponding to the diabetes sequelae to which the prognoses relate. In other embodiments, the entire simulation model comprises so-called Markov chains with transition probabilities between the individual states whose numeral values are taken from important diabetes studies. These studies form the evidence-base of the system (e.g. Accu-Chek Mellibase®). They are typically updated at regular intervals and are evaluated by a standardized method for evaluating the degree of evidence according to “MERGE” before incorporation into the model. The control logic is configured to communicate with the client and is configured for data control within the server. A standard data base may be used to store the query data directed to the system and the results calculated on this basis.

The modular client subcomponent is configured to use the server (Web Service) to generate the risk and potential reports. It typically comprises various modules where each module represents an individual process e.g. data input, calculation and PDF generation. Where possible and appropriate, all modules can be automated. The data base is at the center. It stores the various intermediate stages until the risk and potential reports are completed, and the various modules communicate with one another via this data base.

The manual input of reports can take place concurrently on several computers. Report data are typically sent to the modular client in an electronic form. For this purpose a special CSV format is defined. Applications which write the report data in this CSV format in a predefined directory can be added to input or transmit the report data to the modular client. The data import is not only limited to CSV formats but can be carried out using all of the formats known in the prior art. The module for data import detects when new report data are ready for importing. The report data may then be automatically imported and subsequently archived. If they are complete, the imported reports are immediately released for calculation. Reports released for calculation are then converted into the internal XML format and transferred to the server for calculation. A functioning inter net connection is typically used for this. This can also take place in any other format known from the prior art. After the calculation process, the print out of the risk and potential reports starts automatically.

### BRIEF DESCRIPTION OF THE FIGURES

The following detailed description of the embodiments of the present invention can be best understood when read in conjunction with the following drawings, where like structure is indicated with like reference numerals and in which:

FIG. 1 shows an embodiment of a tabular representation of various influencing factors in relation to the current value, personal target value, long-term target value and attained personal target value.

FIG. 2 shows an embodiment of a bar diagram which shows the deviation of influencing factors from the respective personal target value and corresponding face symbols which evaluate the change compared to the last examination.

FIGS. 3a-3e show an embodiment of horizontal bar diagrams used to visualize the potentials for five different clinical pictures calculated from the current risk status, the short-term target risk and long-term target risk.

FIGS. 4a-4f show an embodiment of graphic representations illustrating the development of absolute risk parameters and personal target values over time.

FIGS. 5a-5e show an embodiment of graphic representations of the risk development of a patient for five different clinical pictures in relation to personal and long-term target values.

Skilled artisans appreciate that elements in the figures are illustrated for simplicity and clarity and have not necessarily been drawn to scale. For example, the dimensions of some of the elements in the figure may be exaggerated relative to other elements to help improve understanding of the embodiment(s) of the present invention.

In order that the present invention may be more readily understood, reference is made to the following examples, which are intended to illustrate the present invention, but not limit the scope thereof.

### DETAILED DESCRIPTION OF EMBODIMENTS OF THE PRESENT INVENTION

The following description of the preferred embodiment is merely exemplary in nature and is in no way intended to limit the present invention or its application or uses.

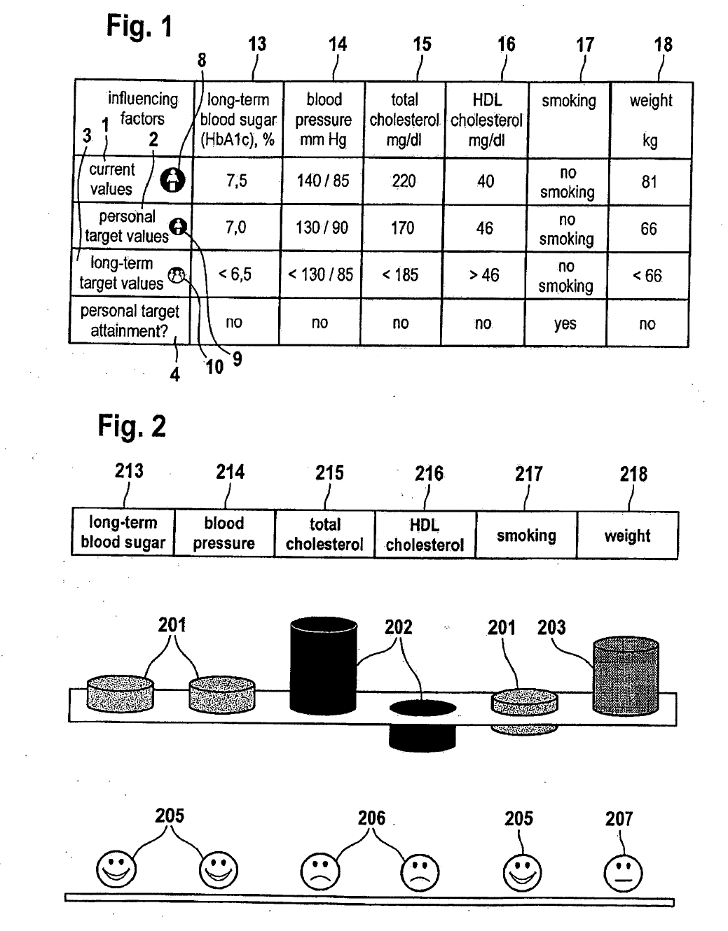

FIG. 1 shows six important influencing factors such as long-term blood sugar (HbAlc) 13, blood pressure 14, total cholesterol 15, HDL cholesterol 16, smoking 17 and weight 18. The current values 1, personal target values 2, long-term target values 3 and the personal target attainment 4 are entered into a system according to the present invention for these influencing factors. The values for the various values may be color-coded (not shown in the drawings). Thus, for example, the current values I may be marked in blue, values for the personal target value 2 in green, long-term target values 3 in light grey and the attained target values 4 may be shown in dark grey. Symbols are used for this in the same colors which represent the current value 8 with a blue symbol, the personal target value 9 with a green symbol and the foot amputation 10 with a light grey symbol. This table displayed by a system according to the present invention gives the patient an overview of the numbers for the current values of the most important influencing factors, his or her personal target values as well as his or her long-term target values and whether these target values have already been achieved.

In one embodiment, the table of FIG. 1 is converted into a diagram such as shown in FIG. 2. This shows the previously identified six important influencing factors as bar diagrams whereby the light-grey middle line (blue-green in the original) shows the personal target value. The dark-grey column 203 (green in the original) shows that the personal target value has been attained, a light-grey column 201 (yellow in the original) denotes a slight deviation (deviation of up to 10%) from the personal target value, whereas a black column 202 (red in the original) denotes a drastic deviation (more than 10% deviation) from the personal target value. In this case deviations can appear for values that are too high for one parameter or values that are too low for one parameter. Hence a value that is too high is disadvantageous for the parameters long-term sugar 213, blood pressure 214, total cholesterol 215, smoking 217 or weight 218. Thus a negative deviation is shown by a column above the target value. In contrast, high values of the parameter HDL cholesterol 216 are assessed as positive, which is why a non-attainment of the target value is shown by a column below the target value. An exact attainment of the target value is shown by a column which shows a small green column below as well as above the target value line. The column 201 in FIG. 2 for the parameter smoking 217 shows that the target value has been reached. This graphic should illustrate to the patient which influencing factors he or she should improve (red column) and which targets he or she has already reached (green column) or nearly reached (yellow column). Moreover, from the first subsequent report onwards the change compared to the values obtained before is shown with the aid of face symbols that show a smiling 205, a frowning 206 and a neutral face 207. Thus a frowning face appears when a negative change has occurred, a smiling face appears when an improvement occurs and a neutral face appears when the values are unchanged.

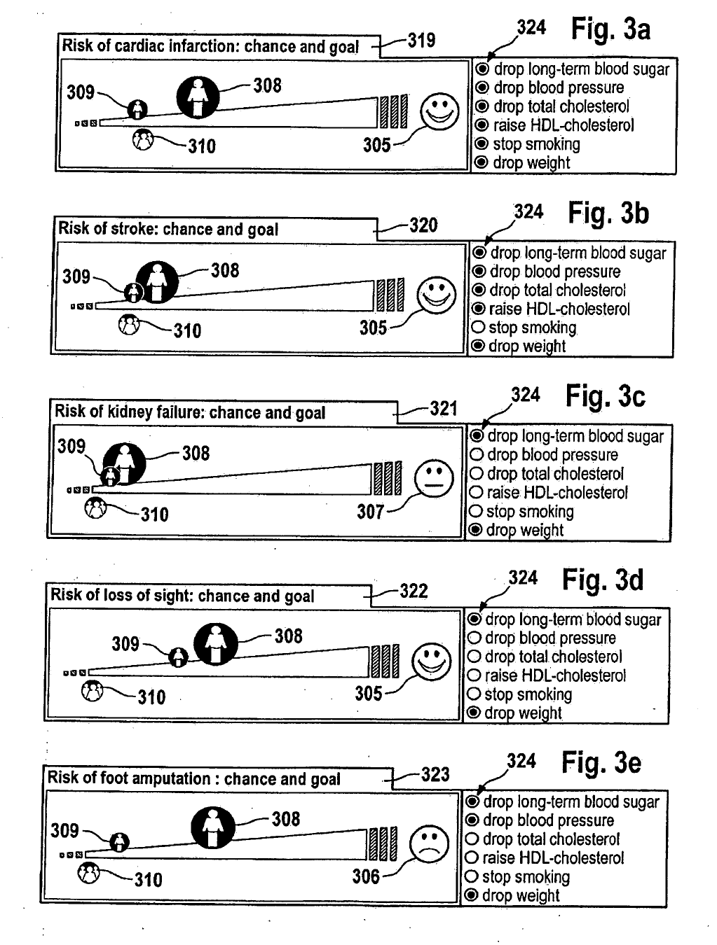

Values from the table in FIG. 1 are used to determine the risk and improvement potential of the patient for various diseases. This risk and potential are shown graphically by a system according to the present invention as a risk status and potential in FIG. 3 for five different clinical syndromes. The five different diseases are cardiac infarction 319, stroke 320, kidney failure 321, loss of sight 322 and foot amputation 323. Three different symbols are used for this. The figure symbols for the potential calculated from the current risk status 308, the potential calculated from the personal short-term target risk status 309 and the long-term target risk 310 are used to make it clear to the patient how high his or her current potential is for reaching the long-term target risk. In this regard, a bar diagram which is arranged horizontally and shows an increase in risk from left to right is used in embodiments of the system for each clinical syndrome. The two symbols for the potential of the current risk status 308 and the potential from the short-term target risk status 309 are arranged above the bar whereas the symbol for the long-term target risk 310 is arranged below the bar. The reason for this is that a different scale is used for the symbols 308 and 309 than for the symbol 310. The potential of the current risk status or the short-term target risk status may be calculated as follows: (current risk—long-term target risk)/current risk or (short-term target risk—long-term target risk)/short-term target risk. The symbol for the long-term target risk 310 is attached below the bar. On a scale of 0 to 30% it shows the magnitude of the absolute risk for contracting the respective disease for the group of people who fulfil the guideline values. The development potential is all the more larger the further the symbol of the current risk status 308 is located to the right. The left border of the bar shows a zero potential for lowering the current risk status in relation to the long-term target risk whereas the right border indicates a 100% potential for lowering the current risk status in relation to the long-term target risk. The same scale applies to the short-term target risk status 309. A smiling 305, neutral 306 or frowning 307 face symbol is again attached next to the bar diagram. This shows the patient whether he or she has got closer to his or her short-term target risk status since his or her last visit to the doctor (smiling face 305), whether the distance to the short-term target risk status has remained the same (neutral face 307) or whether the distance to the short-term target risk status has got larger (frowning face 307). In addition to the face symbol there is row 324 of filled and unfilled circles which indicate whether the patient should change various influencing factors or not. This allows the patient to recognize which influencing factors are important for which disease risk and which factors he or she can and should additionally improve in order to influence this disease risk and to reach his or her short-term target risk status. As a result of the manner of visual representation by the system, the absolute long-term target risks are comparable between the individual graphics as well as among the relative improvement potentials.

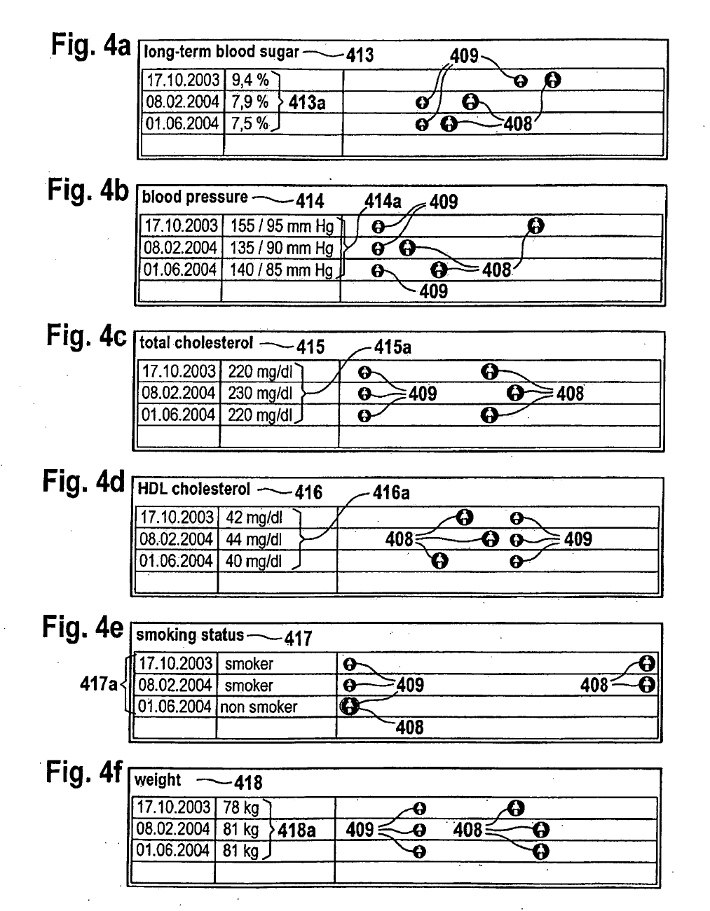

FIG. 4 shows how an embodiment of the system might provide an overview of the development of the various influencing factors over time. In this case it is possible to enter up to four different time points with the corresponding values for the influencing factors 413a-418a. The influencing factors 413-418 are listed vertically and up to four time points and the associated values are recorded to the left in the table 413a-418a. The various figure symbols for the current risk status 408 and the short-term target risk status 409 are shown on the right hand side. In this connection the development of the short-term target risk status and the gap between the current risk status and the respective short-term target risk status are important for the patient. The goal of the patient is to develop towards the short-term target risk status. In doing so the short-term target risk status can change form one time to the next if the patient has reached the short-term target risk status or if he or she is too far removed therefrom. This may be at the discretion of the doctor or patient. In this case the short-term target risk status 409 can either have a lower value than the current risk status 408 as in the case of long-term blood sugar 413, blood pressure 414, total cholesterol 415, smoking status 417 and weight 418 or have higher values as in the case of HDL cholesterol 416.

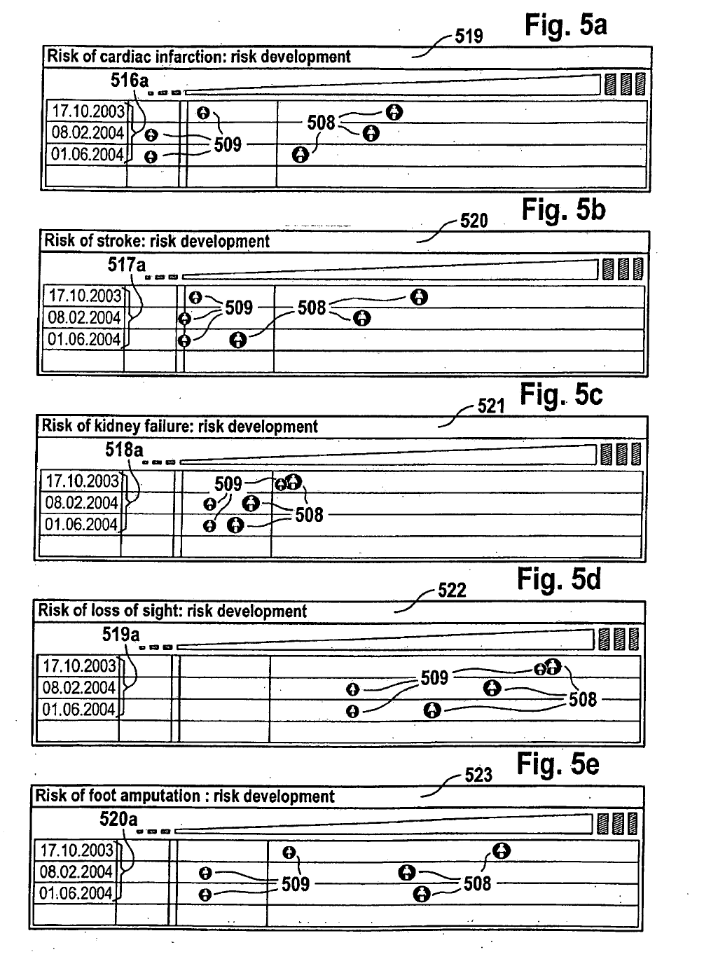

A similar bar diagram to FIG. 4 is shown in FIG. 5 as another embodiment of the system configured to show the patient the time course of risk development for the various clinical pictures. For this purpose the five different clinical pictures cardiac infarction 519, stroke 520, kidney failure 521, loss of sight 522 and foot amputation 523 are arranged one beneath the other. In this case it is also again possible to show the current risk values and the target risk values in relation to the long-term target risk (grey vertical bars) at four different times 516a-520a. FIG. symbols are again used for the current risk status 508 and the short-term target risk status 509. A horizontal bar is shown above each table for each risk which increases in size from left, small risk, to right, large risk. This enables the patient to monitor his or her development over a long time period. In doing so he or she can see the magnitude of the gap that still remains to his or her long-term target risk and whether his or her risk for individual clinical pictures has improved or deteriorated.

The features disclosed in the above description, the claims and the drawing may be important both individually and in any combination with one another for implementing the invention in its various embodiments.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in; a change in the basic function of the subject matter at issue.

Having described the present invention in detail and by reference to specific embodiments thereof, it will be apparent that modification and variations are possible without departing from the scope of the present invention defined in the appended claims. More specifically, although some aspects of the present invention are identified herein as preferred or particularly advantageous, it is contemplated that the present invention is not necessarily limited to these preferred aspects of the present invention.

What is claimed is:

1. A system for visualizing the state of health of a patient comprising:
an input unit for reading in patient data, an evaluation unit for evaluating the data and an output unit which outputs the results of the evaluation in a graphical form, wherein the evaluation unit contains a program configured to read in a parameter value of the patient for at least one risk parameter and to calculate a current risk status and to calculate a short-term target risk status for a change of the parameter value.

2. The system according to claim 1, wherein the at least one risk parameter comprises at least one risk parameter that can be influenced by the patient and at least one risk parameter that cannot be influenced by the patient.

3. The system according to claim 1, wherein the at least one risk parameter comprises at least one of long-term blood sugar, blood pressure, cholesterol, smoking and weight of the patient.

4. The system according to claim 1, wherein the current risk status and the short-term target risk status pertain to risk of at least one long-term sequelae.

5. The system according to claim 1, wherein results of the calculation of current risk status and of short-term target risk status are displayed graphically by the output unit.

6. The system according to claim 1, wherein the program is further configured to calculate risks for various ways of life of the patient, and wherein calculation results thereof are displayed by the output unit.

7. The system according to claim 1, wherein the program is further configured to calculate a long-term target risk for the at least one risk parameter, and wherein the current risk status, the short-term target risk status and the long-term target risk are displayed together graphically by the output unit.

8. The system according to claim 7, wherein the output unit graphically displays the current risk status, the short-term target risk status and the long-term target risk using a plurality of charts.

9. The system according to claim 1, wherein the program is further configured to calculate the current risk status and the short-term target risk status for various times such that the graphical form output by the output unit comprises a visualization of the change in the patient's risk over time.

10. The system according to claim 7, wherein the program is further configured to calculate the current risk status, the short-term target risk status and the long-term target risk for various times such that the graphical form displayed by the output unit comprises a visualization of the change in the patient's risk over time.

11. The system according to claim 7, wherein the graphical display of the current risk status, the short-term target risk status and the long-term target risk comprises a plurality of different symbols.

12. The system according to claim 11, wherein the plurality of different symbols are shown on a horizontal bar diagram comprising bars that widen with increasing risk.

13. The system according to claim 11, wherein an improvement of the at least one risk parameter is marked with a positive symbol, a deterioration of the at least one risk parameter is marked with a negative symbol, and no or negligible change of the at least one risk parameter is marked with a neutral symbol at the edge of the bar diagram.

14. The system according to claim 4, wherein the at least one long-term sequelae is selected from the group consisting of cardiac infarction, stroke, kidney failure, loss of sight and foot amputation.
